(19) United States
(12) Patent Application Publication
(10) Pub. No.: US 2008/0233192 A1
(43) Pub. Date: Sep. 25, 2008

(54) Tablets with Improved Dissolution Profile

(76) Inventors: Jose Luis Fabregas Vidal, Barcelona (ES); Nuria Garcia Gonzalez, Barcelona (ES); Pere Guiro Coll, Barcelona (ES); Antoni Masso Carbonell, Barcelona (ES)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/996,797

(22) PCT Filed: Jul. 10, 2006
(86) PCT No.: PCT/EP2006/006718

§ 371 Date: Mar. 6, 2008

(30) Foreign Application Priority Data
Jul. 28, 2005 (ES) P200501852

(51) Int. Cl. A61K 9/36

(52) U.S. Cl. 424/480

(57) ABSTRACT

The present disclosure relates to tablets obtainable by compression of a mixture comprising at least 5% by weight of a diluent and granules which comprise a very slightly soluble drug, wherein the granules consist of a core of saccharose beads having (prior to coating) a mean particle size greater than 150μ and a coating layer comprising the very slightly soluble drug and at least one film-forming substance of high-molecular weight, as well as a process for manufacturing such tablets wherein the granules consist of a core saccharose beads having (prior to coating) a mean particle size greater than 150μ and a coating layer comprising the very slightly soluble drug and at least one film-forming substance of high-molecular weight for the manufacture of tablets by compression of a mixture comprising said granules and at least 5% by weight of a diluent.

The present invention relates to tablets featuring good dissolution and absorption of very slightly soluble drugs contained therein and a production process thereof.

It has been observed that it is not sufficient for tablets to disintegrate quickly to guarantee a fast dissolution of very slightly soluble drugs contained therein. In effect, when tablets are formed by direct compression or wet compression by using relatively very slightly soluble drugs, the tablets so obtained are very often showing a poor dissolution profile, i.e. they dissolve too slowly.

This is a drawback since fast dissolution of tablets is a prerequisite to achieve a good profile of absorption of the drug contained therein.

### SUMMARY OF THE INVENTION

Accordingly, an object of the present invention is to provide tablets comprising a very slightly soluble drug featuring good dissolution of such drug.

In view of the foregoing circumstances, the present inventors have carried out an extensive investigation with a view toward obtaining tablets containing a very slightly soluble drug and yet having a good dissolution profile.

As a result, it has been found that tablets with a good dissolution profile and/or absorption can be obtained by direct compression of a mixture comprising at least 5% by weight of a diluent and granules which comprise a very slightly soluble drug, wherein the granules consist of a core of saccharose beads having (prior to coating) a mean particle size greater than 150μ and a coating layer (preferably a single coating layer) comprising the very slightly soluble drug and at least one film-forming substance of high-molecular weight.

Accordingly in one aspect the present invention provides tablets obtainable by compression of a mixture comprising at least 5% by weight of a diluent and granules which comprise a very slightly soluble drug, wherein the granules consist of a core of saccharose beads having (prior to coating) a mean particle size greater than 150μ and a coating layer comprising the very slightly soluble drug and at least one film-forming substance of high-molecular weight. It is preferred that the saccharose beads are coated with a single coating layer.

The present invention also provides a process for producing tablets comprising very slightly soluble drugs, which comprises dispersing the very slightly soluble drug into a solution of at least one film-forming substance of high-molecular weight to form a solution or suspension; coating saccharose beads of a mean particle size greater than 150μ with the above-mentioned drug dispersion; optionally drying the coated beads; mixing the coated beads with standard ingredients for compression which ingredients comprise at least 5% by weight of a diluent and tabletting the mixture to form tablets.

It is another aspect of the present invention the use of granules consisting of a core of saccharose beads having (prior to coating) a mean particle size greater than 150μ and a coating layer comprising the very slightly soluble drug and at least one film-forming substance of high-molecular weight for the manufacture of tablets by compression of a mixture comprising said granules and at least 5% by weight of a diluent.

Tablets obtained employing the production process according to the present invention, have good dissolution.

As hereinabove described the tablets of the present invention are manufactured by compression of a mixture comprising at least 5% of a diluent and coated saccharose beads. The coated saccharose beads are produced by coating saccharose beads having a mean particle size greater than 150μ with a solution or suspension of the very slightly soluble drug in a solution of at least one film-forming substance. The saccharose beads may be coated using any conventional coating process such as fluid bed coating, pan coating or the like.

As use herein the term saccharose beads designates saccharose spheres as defined in the US Pharmacopoeia (under the heading Sugar Spheres) which comprise from 62.5% to no more than 91.5% by weight of saccharose calculated on the dried basis. The beads often comprise starch as the second major component thereof. The beads are available on the market in a variety of particle sizes ranging from 180 to 1700 microns. It has been found that saccharose beads having particle size larger than 150μ, particularly between 200 and 425μ and most preferably between 250 and 355μ are particularly well adapted for the manufacture of the tablets of the present invention.

Preferably, saccharose beads can be used in an amount 5-20 times by weight as much as the very slightly soluble drug, with 8-18 times by weight being preferred and 11-14 times by weight being particularly preferred.

In an embodiment of the invention the saccharose beads have a mean particle size in the range comprised between 200 and 400μ.

In another embodiment the weight ratio of saccharose beads to very slightly soluble drug is comprised between 8 and 18.

In still another embodiment of the invention the coating layer comprises a water-soluble cellulose derivative.

As use herein the term very slightly soluble drug designates drugs which have a solubility in water of not more than 0.1 mg/ml in the conditions defined in section 5.11 of the European Pharmacopoeia 5.0.

As described above the saccharose beads are coated with the very slightly soluble drug dispersed or dissolved in a solution of a film-forming substance. It is preferred that the solution is an aqueous solution.

As used herein the term film-forming substance designates a water-soluble high-molecular weight substance which has the capacity of forming coating films. By high-molecular weight it is understood a molecular weight of more than 300 Daltons. It is preferred that the film-forming substance used does not significantly delay the dissolution of the very slightly soluble drug. Thus, it is highly preferred not to use enteric coating film-forming substances. For similar reasons it is preferred that the only coating layer of the saccharose beads is the one obtained by the coating composition comprising the very slightly soluble drug dispersed or dissolved in a solution of a film-forming substance.

Film-forming substances are added to the solution used to disperse or dissolve the very slightly soluble drug in order to obtain the composition used for coating the saccharose beads. Examples of film-forming substances are acacia gum, arabic gum, pectin, gelatin, polyvinylpyrrolidone, starch (paste and pre-gelatinized), sodium alginate and alginate derivatives, tragacanth, soluble celluloses like methylcellulose, ethylcellulose, carboxymethylcellulose, hydroxypropylmethylcellulose and hydroxypropylcellulose; polyethyleneglycols; polyvinylalcohol polymers such as polyvinylalcohol and polyvinylacetate and acrylic polymers such as polymethacrylates. In a preferred embodiment the solution comprises from 0.1 to 30 % wt. of the film-forming substances. The film forming solution may comprise one or more than one film-forming substance. It is a preferred embodiment that the solution comprises only one film-forming substance.

In addition to the film-forming substances, the solution in which the very slightly soluble drug is dispersed or dissolved, may contain film adjuvants helping the formation of a cohesive and plastic coating layer. Examples of such adjuvants are plasticizers such as liquid polyethylenglycols and triethylcitrate.

In addition to the film-forming substances and the film adjuvants, the solution used to disperse or dissolve the very slightly soluble drug may, when desired, also contain a surfactant such as sorbitan fatty acid esters, polyoxyethylene fatty acid esters (i.e. PEG-40 hydrogenated castor oil (Cremophor® RH40)), fatty acid sulfates (i.e. sodium lauryl sulphate) and dioctyl sodium sulfosuccinate; pigments such as titanium dioxide and iron oxides; antiadherents or glidants such as talc and magnesium stearate and antifoaming agents such as silicon oils.

Although it is preferred to use water as a solvent for the preparation of the coating solution, any other solvent can be employed for this purpose without any particular limitation insofar as it is pharmaceutically acceptable and is able to dissolve the water-soluble high-molecular substance therein. Examples of such a solvent include organic solvents such as ethanol, isopropanol and methylene chloride. Among them, water and ethanol are particularly preferred in view of the fact that the final product is a pharmaceutical.

For the production of tablets according to the present invention, the coated saccharose beads are mixed with the diluent and with the rest of optional ingredients. There are no special restrictions to the diluent used in the present invention. Examples of diluents that may be used are organic substances such as saccharides, cellulose powder, microcrystalline cellulose, starch and the like, inorganic substances such as anhydrous calcium phosphate, precipitated calcium carbonate, calcium silicate, dibasic calcium phosphate and the like. The mixing of the saccharose beads and the diluent can be conducted preferably in a tumbler, a stirring-type mixer such as vertical granulator or a twin-cylinder mixer.

In addition to the diluents described in the previous paragraph other conventional ingredients may be added to the coated saccharose beads prior to tabletting the resulting mixture. Non-limiting examples of these ingredients are lubricants such as talc and sodium stearyl fumarate; disintegrants such as crospovidone; binders such as polyvinylpyrrolidone, polyethytleneglycols, hydroxypropylmethylcellulose, methylcellulose, ethylcellulose and starch; flavour; taste masking agents; colouring agents and the like.

The mixture obtained is then subjected to compression, whereby tablets of the present invention can be obtained. It is preferred to use direct compression.

For the very slightly soluble drug to be used in the present invention there are no particular limitations as long as it is a substance which is used as a pharmaceutical active ingredient and it has a solubility in water of not more than 0.1 mg/ml in the conditions defined in section 5.11 of the European Pharmacopoeia 5.0. Examples of pharmaceutical active ingredients include: sedative hypnotics, sleep inducers, anti-anxiety drugs, anti-epileptics, anti-depressants, anti-Parkinson drugs, psychoneural drugs, drugs acting on the central nervous system, local anesthetics, skeletal muscle relaxants, autonomic nervous system drugs, anti-fever analgesics anti-inflammatory drugs, anti-convulsants, anti-vertigenous drugs, cardiac drugs, drugs for arrhythmia, diuretics, blood pressure lowering drugs, vasoconstrictors, vasodilators, drugs for circulatory organs, hyperlipidemia drugs, respiratory stimulant, anti-tussive expectorants, bronchodilators, stegnotic, peptic ulcer drugs, stomach digestive drugs, antacids, laxatives, choleretics, drugs for the digestive tract, adrenal hormone drugs, hormone drugs, urinary tract drugs, vitamins, hemostatic drugs, liver drugs, gout treatment drugs, drugs for diabetes, anti-histamines, antibiotics, antibacterial agents, anti-malignant tumour drugs, chemotherapy drugs, multi-purpose cold medicines, tonic medicines, osteoporosis drugs, and the like. Preferred very slightly soluble drugs to be used in the present invention are selected from the group consisting of 3-(2,4-difluorophenoxy)-2-(4-methanesulphonylphenyl)-6-methylpyran-4-one, 4-diphenylmethoxy-1-[3-(4-tert-butylbenzoyl)propyl]piperidine, 3-(4-chlorophenyl)-1-propylxanthine, 3-[2-(dimethylamino)ethyl]-5-(pyrrolidin-1-ylsulfonylmethyl)-1H-indole and 2-[4-[4-[4-(diphenylmethoxy)piperidin-1-yl]butyryl]phenyl]-2-methylpropionic acid.

The amount of diluent used in the present invention is higher than 5% and adjusted as needed in accordance with the dose of the drug and/or size of the tablet. The amount added is adjusted as needed so that a tablet of the desired size is obtained by increasing the amount added when the dose of drug is small, reducing the amount added when the dose of drug is large, and the like, but it is usually 5 to 99.5 w/w %, preferably 20 to 95 w/w %, per tablet weight.

### EXAMPLES

The present invention will next be described in detail by the following examples. It should however be borne in mind that this invention is by no means limited to or by the examples.

The ingredients used in the following examples are described below:

Very slightly soluble drug 1: 3-(2,4-difluorophenoxy)-2-(4-methanesulfonylphenyl)-6-methylpyran-4-one

Very slightly soluble drug 2: ebastine (4-Diphenylmethoxy-1-[3-(4-tert-butylbenzoyl)propyl]piperidine)

Saccharose: Saccharose/maize starch spheres having a particle size distribution between 180 and 250 microns

Sepifilm 003 (marketed by SEPPIC): Pregranulated specialty product for aqueous coating comprising 45-55% HPMC (hydroxypropylmethylcellulose), 35-45% MCC (microcrystalline cellulose) and 8-12% Macrogol-40-stearate.

Polyglycol 6000 P: Solid polyethylene glycol with an average molecular weight of 6000

Cremophor RH 40 (marketed by BASF): A non-ionic emulsifying agent produced by allowing 1 mole of castor oil to react with 40-45 moles of ethylene oxide

AVICEL PH 102 (marketed by FMC): a microcrystalline cellulose

EMCOMPRESS (marketed by JRS Pharma): Dibasic calcium phosphate dihydrate

Poliplasdone XL (marketed by ISP): crospovidone meeting Ph Eur pharmacopeial Monograph

Pruv (marketed by JRS Pharma): sodium stearyl fumarate

Formulation 1

| | | Amount |
|---|---|---|
| | Ingredient | (in mg) |
| 1 | Very slightly soluble drug 1 | 25 |
| 2 | Saccharose | 337 |
| 3 | Sepifilm 003 | 8.6 |
| 4 | Polyglycol 6000 P | 1.6 |
| 5 | Cremophor RH40 | 5 |
| 6 | Avicel PH 102 | 153.5 |
| 7 | Encompress | 15 |
| 8 | Poliplasdone XL | 35.3 |
| 9 | Talc | 12 |
| 10 | Pruv | 7 |
| | TOTAL | 600 |

### Example 1

Procedure

Out of the above ingredients, Polyglycol 6000P and Cremophor RH40 were dissolved in water. The very slightly soluble drug 1 and Sepifilm 003 were mixed and added to the above mentioned solution. The resulting suspension was homogenized during 1 minute in an Ultra-Turrax homogenizer working at 4000 r.p.m. The resulting suspension was kept stirred until used in the bed coating step described below.

A Uni-Glatt fluid bed coater with Wurster system was equipped with 0.8 mm nozzles. The apparatus was allowed to work while empty for 5 minutes in order to pre-heat it. The apparatus was then sttoped and the saccharose beads were placed in the basket. The apparatus was started again and coating was made using the previously prepared suspension under the following working conditions:

Spraying pressure: 1.25 bars

Aspiration floodgate: 35%

Inlet temperature: 35-45° C.

Outlet temperature: 45-55° C.

Peristaltic pump flow rate: 10.4 g/min/kg

The coated beads were dried during 3 minutes at 40° C. and then sieved using a mesh of 30 ASTM. 378.2 mg of the sieved granules and the ingredients 6 to 10 were mixed in a tumbler and then compressed in a KORSCH machine equipped with 17×17.5 mm biconcave punches at a hardness of 60-80 N.

### Comparative Example 2

The same ingredients and proportions of example 1 are used but the procedure is changed in that the very slightly soluble drug 1 is added, together with ingredients 6 to 10, to the saccharose beads once they have been coated with a composition consisting of ingredients 3 to 5.

### Comparative Example 3

The same ingredients and proportions of example 1 are used but the saccharose beads are ground to an average particle size of 30μ and then granulated with ingredients 1, 3, 4 and 5 in a Lödige granulator.

### Comparative Example 4

The same ingredients, proportions and procedure of example 1 are used with the exception that the saccharose beads are ground to an average particle size of 30μ once they have been coated with ingredients 3, 4 and 5 and prior to mixing with the rest of ingredients.

### Comparative Example 5

The same ingredients and proportions of example 1 are used but the very slightly soluble drug 1 is mixed with the saccharose beads once they have been coated with a composition consisting of ingredients 3 to 5. Subsequently ingredients 6 to 10 are mixed with the resulting composition.

Only the procedure followed in example 1 results in granules which consist of a core of saccharose beads having (prior to coating) a mean particle size greater than 150μ and a coating layer comprising the very slightly soluble drug and a film-forming substance of high-molecular weight. In the rest of the examples either the integrity of the saccharose beads has been destroyed or the very slightly soluble drug does not form part of the layer coating the beads.

Formulation 2

| | | Amount |
|---|---|---|
| | Ingredient | (in mg) |
| 1 | Very slightly soluble drug 2 | 20 |
| 2 | Saccharose | 337 |
| 3 | Sepifilm 003 | 8.6 |
| 4 | Polyglycol 6000 P | 1.6 |
| 5 | Cremophor RH40 | 5 |
| 6 | Avicel PH 102 | 158.8 |
| 7 | Encompress | 15 |
| 8 | Poliplasdone XL | 35 |
| 9 | Talc | 12 |
| 10 | Pruv | 7 |
| | TOTAL | 600 |

### Example 6

The tablets were prepared following the process described in example 1 using the formulation 2.

### Comparative Example 7

The same ingredients and proportions of example 6 are used but the procedure is changed in that the very slightly soluble drug 2 is added, together with ingredients 6 to 10, to the saccharose beads once they have been coated with a composition consisting of ingredients 3 to 5.

Dissolution Test

The dissolution test is performed on the tablets to be tested using Apparatus 2 as described in Chapter 711 (Dissolution) of the US Pharmacopeia under the following conditions:
Stirring speed: 50 r.p.m.
Temperature: 37° C.±0.5° C.
Solvent: Acetate buffer pH=3.8 with 0.25% SDS (Sodium dodecyl sulphate) when the very slightly soluble drug is 3-(2,4-difluorophenoxy)-2-(4-methanesulfonylphenyl)-6-methylpyran-4-one and HCl 0.01 N when the very slightly soluble drug is 4-Diphenylmethoxy-1-[3-(4-tert-butylbenzoyl)propyl]piperidine
Volume of solvent: 900 ml when the very slightly soluble drug is 3-(2,4-difluorophenoxy)-2-(4-methanesulfonylphenyl)-6-methylpyran-4-one or 1000 ml when 4-Diphenylmethoxy-1-[3-(4-tert-butylbenzoyl)propyl]piperidine
Total time: 60 min
Sampling interval: 5 min

To establish the dissolution profile aliquots are taken at regular intervals of 5 minutes from the vessels and the concentration of the very slightly soluble drug therein is determined by spectrometry using a wavelength of 280 nm when the very slightly soluble drug is 3-(2,4-difluorophenoxy)-2-(4-methanesulfonylphenyl)-6-methylpyran-4-one and 258 nm when the very slightly soluble drug is 4-Diphenylmethoxy-1-[3-(4-tert-butylbenzoyl)propyl]piperidine.

Disintegration Test

The disintegration test is performed following the procedure described in Chapter 2.9 (Disintegration of tablets and capsules) of the EP Pharmacopeia 5.0.

Results

| | DISSOLUTION AT 10 min | DISINTEGRATION TIME |
|---|---|---|
| EXAMPLE | (in %) | (in s) |
| 1 | 83.9 | 31 |
| COMP 2 | 57.0 | 77 |
| COMP 3 | 69.5 | 182 |
| COMP 4 | 52.8 | 292 |
| COMP 5 | 58.3 | 76 |
| 6 | 78.3 | 33 |
| COMP 7 | 55.0 | 71 |

1. A tablet obtainable by compression of a mixture comprising at least 5% by weight of a diluent and granules which comprise a very slightly soluble drug, wherein the granules comprise a core of saccharose beads having (prior to coating) a mean particle size greater than 150μ and a coating layer comprising the very slightly soluble drug and at least one film-forming substance of high-molecular weight.

2. A tablet according to claim 1, wherein the saccharose beads have a mean particle size ranging from 200 to 400μ.

3. A tablet according to claim 1, wherein the weight ratio of saccharose beads to the very slightly soluble drug ranges from 8 to 18.

4. A tablet according to claim 1, wherein the coating layer comprises a water-soluble cellulose derivative.

5. A process for manufacturing a tablet comprising the steps of:
i) dispersing or dissolving a very slightly soluble drug into a solution comprising at least one film-forming substance of high-molecular weight;
ii) coating saccharose beads of a mean particle size greater than 150μ with the above-mentioned drug dispersion or solution;
iii) optionally drying the coated beds;
iv) mixing the coated beds with standard ingredients for compressions; wherein the ingredients comprise at least 5% by weight of a diluent; and
v) tabletting the mixture to form tablets.

6. The process according to claim 5, wherein the saccharose beads have a mean particle size ranging from 200 to 400μ.

7. The process according to claim 5, wherein the weight ratio of saccharose beads to the very slightly soluble drug ranges from 8 to 18.

8. The process according to claim 5, wherein the coating layer comprises a water-soluble cellulose derivative.

9. (canceled)

10. (canceled)

11. (canceled)

12. (canceled)
